(19) United States
(12) Patent Application Publication
(10) Pub. No.: US 2010/0048696 A1
(43) Pub. Date: Feb. 25, 2010

(54) AGRICULTURAL DISEASE CONTROL AGENT

(75) Inventors: Kazuo Sumi, Chiba (JP); Kazuyoshi Koike, Chiba (JP); Shinji Ito, Chiba (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: Idemitsu Kosan Co., Ltd., Chiyoda-ku ,Tokyo (JP)

(21) Appl. No.: 12/522,760

(22) PCT Filed: Jan. 11, 2008
(86) PCT No.: PCT/JP2008/050250

§ 371 Date: Jul. 10, 2009

(30) Foreign Application Priority Data
Jan. 12, 2007 (JP) 2007-004892

(51) Int. Cl. A01N 43/08; C07C 57/145; C07D 307/36; A01N 37/06; A01P 1/00

(52) U.S. Cl. 514/473; 562/595; 549/262; 514/574

(57) ABSTRACT

The present invention provides a disinfectant comprising as an active ingredient maleic acid and/or maleic anhydride.

### TECHNICAL FIELD

The present invention relates to a disinfectant, and more particularly, to a disease control agent for disinfecting agricultural materials. More specifically, the present invention relates to a disease control agent for disinfecting a cutting knife.

### BACKGROUND ART

As microbicides used as disinfectants in the agricultural field, Chemichlon G (calcium hypochlorite) manufactured by Nippon Soda Co., Ltd. and Ichiban (benthiazole emulsion) manufactured by Otsuka Chemical Co., Ltd. are available (Patent Document 1 and Non-patent Document 1). Chemichlon G contains active chlorine at 70% and its microbicidal performance is derived from chlorine which is a toxic gas. Therefore, Chemichlon G has shortcomings that it is extremely harmful for human health and disinfected materials can not be used unless being exposed to sunlight for one day to two days until chlorine on the disinfected materials disappears. Since Ichiban has toxicity to fish, Ichiban can not be used around a fish breeding pond and the like. In addition, Ichiban is also harmful for human body. Thus, devices such as protection glasses, a face guard, rubber gloves, and boots for not exposing skin are necessary when Ichiban is used. Further, it is necessary to put 1% calcium hydroxide into it after use, and to expose it to the sunlight for several days to decompose an active ingredient. As described above, the conventional disinfectants for the materials have had problems to be overcome.

Aforementioned Chemichlon G has been widely used as a disinfectant for a cutting knife used for cutting seed tubers of potatoes. A cutting knife farm tool is used for cutting a seed tuber of the potato into 2 to 4 of small pieces before the seed tuber is planted. This work is still carried out manually using the cutting knife (kitchen knife or the like) in Hokkaido, which is a production district of a large number of potatoes. Meanwhile, as serious diseases of the potato, blackleg, soft rot, and the like caused by bacteria belonging to the genus Erwinia have been known. It has been proved that these diseases transmit from a diseased potato to a healthy potato through the cutting knife (kitchen knife, cutter, and the like) in cutting the seed tuber (Non-patent Document 2).

For the purpose of controlling such diseases, a 500 times solution of corrosive sublimate has been used as a disinfectant for the cutting knife farm tool used for the potato seed tuber. However, organic mercury is an active ingredient in the corrosive sublimate, which has been prohibited to use due to its strong toxicity, and a 10 times solution of aforementioned Chemichlon G has been used as its alternative. The 10 times solution of Chemichlon G is a very high concentration, which corresponds to 50 to 200 times concentration of solutions usually used, but perfect sterilization is impossible without using the solution at such a very high concentration when the sterilization of the cutting knife or the like is required in a short period of time. Therefore, there is no choice other than to work under a very bad environment in which chlorine, a toxic gas, is generated in a working site. Thus, an alternative agent has been strongly desired.

On the other hand, an industrial microbicide containing maleic acid or anhydride thereof as an active ingredient has been known as an industrial microbicide (Patent Document 2). However, no example of using maleic acid or anhydride thereof for the agricultural material or the like has been known.

### Patent Document 1: JP 05-155604 A

### Patent Document 2: JP 64-50803 A

Non-patent Document 1: Summary of toxicity studies on pesticides, Benthiazole. Journal of Pesticide Science, Vol. 12, No. 2, Pages 343-345 (May, 1987)
Non-patent Document 2: Propagation way of potato blackleg, Bulletin of Hokkaido Prefectural Agricultural Experiment Station 28., Pages 62-69 (1973)

### DISCLOSURE OF THE INVENTION

As described above, Ichiban and Chemichlon G which are conventional agricultural disinfectants had a problem that the material after being disinfected can not be used immediately, in addition to their harmfulness to human body and their load to the environment. It has also been necessary to treat a residual solution of the disinfectant after disinfection.

The present invention has been made from the above perspectives, and it is an object of the present invention to provide an agricultural disease control agent which has an excellent disease control effect, can be used safely and easily, and has less influence on the environment.

As a result of the extensive study for solving the above-mentioned problems, the inventors of the present invention have found that maleic acid and/or maleic anhydride have/has a control effect on agricultural diseases as the agricultural disease control agent, and have completed the present invention based on those findings.

That is, the summary of the present invention is as follows.

(1) A disinfectant comprising as an active ingredient maleic acid and/or maleic anhydride, wherein a content of the maleic acid and/or the maleic anhydride is 1% by mass or more with respect to an entire disinfectant.

(2) The disinfectant according to (1), which is an agent for controlling an agricultural disease.

(3) The disinfectant according to (1), which is an agent for disinfecting a cutting knife.

(4) The disinfectant according to any of (1) to (3), wherein the disease is caused by any one of bacteria, filamentous fungi, actinomycetes, and viruses.

(5) A method of controlling a disease, comprising treating a cutting knife with the disinfectant according to (3).

(6) The method of controlling a disease according to (5), wherein the disease is blackleg, soft rot, or ring rot of potatoes.

(7) The method of controlling a disease according to (6), wherein the blackleg or soft rot is caused by any one of Erwinia chrysanthemi and Erwinia cartovora var. cartovora, and the ring rot is caused by Clavibacter michiganensis.

(8) A method of controlling a disease, comprising disinfecting an agricultural material with maleic acid and/or maleic anhydride.

(9) Use of maleic acid and/or maleic anhydride for disinfection.

The agricultural disease control agent for disinfecting a material of the present invention (hereinafter sometimes referred to as a “material disinfectant of the present invention” simply) has lower harmfulness to human body compared with conventional disease control agents, allows the material to be used immediately after being disinfected, and can be treated under a mild condition without generating heat if alkali such as calcium carbonate is used.

Cutting seed tubers of potatoes in Hokkaido is carried out typically during a period when the temperature is very low (2 to 8° C.) in March and April. Since seed tubers in an enormous amount are cut, it is necessary that the kitchen knife after cutting the seed tuber be disinfected within at most about 5 seconds. When a potato infected with Erwinia bacteria is cut, the Erwinia bacteria of a high concentration (10⁷ to 10¹⁰ cfu/mL) adhere to the cutting knife, and the material disinfectant of the present invention can disinfect such pathogens of a high concentration in an extremely short time of about 5 seconds or less.

According to the present invention, it is possible to provide the agricultural disease control agent which is excellent in a disease control effect, can be used safely and easily, and has less influence on the environment.

### BRIEF DESCRIPTION OF THE DRAWINGS

FIG. 1 is a graph illustrating disinfection performance of organic acids on blackleg pathogens.

FIG. 2 is a graph illustrating disinfection performance of organic acids on ring rot pathogens.

FIG. 3 is a graph illustrating results of a disinfection test of cutting knives in blackleg.

FIG. 4 is a graph illustrating results of a disinfection test of cutting knives in ring rot.

### BEST MODE FOR CARRYING OUT THE INVENTION

The present invention will be described in detail below. The term “times (solution)” herein represents a unit indicating a degree of dilution. For example, a “10 times solution” means the solution where 1 kg (solid) or 1 L (liquid) of a substance is dissolved in 9 L of a solvent. Such an expression is known as a technical knowledge in the agricultural chemical field.

(Disease Control Agent of the Present Invention)

A disease control agent of the present invention is the disease control agent which exerts an excellent microbicidal action upon pathogens which cause diseases in agricultural and horticultural crops such as grain crops and vegetables.

The disease control agent of the present invention contains maleic acid and/or maleic anhydride as active ingredients. In the present invention, maleic acid and maleic anhydride may be used alone or may be used appropriately in mixture.

Maleic acid and/or maleic anhydride used in the present invention is widely commercially available, and such commercially available products can be used without being particularly limited.

The disease control agent of the present invention can also be formulated by ordinary methods except that maleic acid and/or maleic anhydride is contained. That is, the disease control agent of the present invention may be formulated together with various optional ingredients if necessary. Dosage forms include liquids, powders, granules, emulsions, oils, capsules, wettable powders, endermic liniments, and flowables. Liquid carriers, solid carriers, surfactants (including emulsifiers, dispersants, and anti-foams), and adjuvants can be used in the formulation.

As the liquid carrier, water, a plant oil, a liquid animal oil, a phosphate buffer, a carbonate buffer, a physiological saline, an organic solvent (such as acetone and DMSO), or the like may be used. Maleic acid has good water solubility and maleic anhydride has poor water solubility, and hence a liquid carrier suitable for each of them may be used. As the solid carrier, natural mineral powders including a cation clay, virofilite clay, bentonite, montmorillonite, kaolin, clay, talc, chalk, quartz, attapulgite, and diatomaceous earth, synthetic mineral powders including silicic acid, alumina, and silicate, and polymer natural substances including a crystalline cellulose, corn starch, gelatin, and alginic acid may be used. One kind of the liquid carriers and/or solid carriers may be used or two or more kinds of them may be mixed and used.

As the surfactant, polyoxyethylene-fatty acid ester, polyoxyethylene-fatty alcohol ether, alkylaryl polyglycol ether, alkyl sulfonate, alkyl sulfate, aryl sulfonate, or the like may be used.

As the adjuvant, carboxymethyl cellulose, polyoxyethylene glycol, glycerin, acacia gum, starch, lactose, and the like may be used.

The disease control agent of the present invention may be maleic acid and/or maleic anhydride themselves, or may be formulated from maleic acid and/or maleic anhydride together with the optional ingredients as described above. On formulation, a concentration of maleic acid and/or maleic anhydride in the disease control agent of the present invention may be any concentration, and, for example, is preferably 0.2 to 100% by mass.

(Method of Using the Disease Control Agent of the Present Invention)

The disease control agent of the present invention exhibits a disease control action upon plants, and can be used as an agricultural disease control agent, specifically, a disinfectant for materials, for example. When the disease control agent of the present invention is used for disinfecting materials, as an application method, any method can be appropriately selected depending on a use form such as a dosage form, a type of the material of interest, and a type of the disease of interest.

A treating method using a material disinfectant includes a method of immersing a material in the material disinfectant of the present invention or a method of spraying the material disinfectant of the present invention in the form of solution on the material.

When the disease control agent of the present invention is used for disinfecting the material, the concentration of maleic acid and/or maleic anhydride is set to preferably a 500 times or less solution (when represented by % by mass, 0.2% by mass or more), more preferably a 250 times or less solution (0.4% by mass or more), still more preferably a 2 to 250 times solution (0.4 to 50% by mass), and particularly preferably a 2 to 100 times solution (10 to 50% by mass). When the disinfectant is a 100 times or less solution, it is possible to completely sterilize the pathogens immediately after disinfection. Even if the concentration exceeds the 100 times, when the disinfectant is the 500 times or less solution, even if the pathogens survive immediately after disinfection, it is possible to induce the complete sterilization of the pathogens by dried and concentrated products of maleic acid and/or maleic anhydride. Because of product design, it is preferable to be diluted to 2 times or more.

When the disease control agent of the present invention is used for disinfecting a material to which pathogens have adhered at an extremely high concentration, or when the material is disinfected in a short period of time, the concentration of maleic acid and/or maleic anhydride in the disease control agent of the present invention is set to preferably a 100 times or less solution (when represented by % by mass, 1% by mass or more), more preferably a 2 to 100 times solution (1 to 50% by mass), and still more preferably a 2 to 10 times solution (10 to 50% by mass). When the disinfectant is a 100 times or less solution, it is possible to sufficiently keep a sterilizing capacity for the pathogens immediately after disinfection and completely sterilize the pathogens in a short period of time. If the complete sterilization in a short period of time, e.g., instantaneously, is considered, it is more preferable to use a 10 times or less solution. In the case of such a use, an immersion time is, for example 10 seconds or less, 5 to 10 seconds in terms of economical viewpoint, and in some cases, can be 5 seconds or less. Because of the product design, it is preferable to be diluted to 2 times or more.

As a specific sterilization method using the material disinfectant of the present invention, for example, the following method can be used. When the agricultural material (e.g., raising seedling boxes (wooden boxes, plastic boxes), raising seedling pots, pot braces, fruit storage boxes, farm tools, cutting knives, rakes, shovels, and the like) is disinfected, maleic acid and/or maleic anhydride is dissolved in tap water to prepare a disinfection solution as a 100 times solution. The raising seedling box or the raising seedling pot is immersed in the disinfection solution of maleic acid (and/or maleic anhydride) for 10 minutes. When the material is the pot brace, the disinfection solution is sprayed on the pot brace using a watering pot. Here the cutting knife may be any of a plow, a sickle, a harrow, a saw, a chopper, a kitchen knife, a knife, a sword, or the like which can cut vegetables, fruits, and grain crops.

In the case of the cutting knife for potatoes, the concentration of maleic acid and/or maleic anhydride in the disease control agent of the present invention is set to preferably a 100 times or less solution (when represented by % by mass, 1% by mass or more), more preferably a 50 times or less solution (2% by mass or more), still more preferably a 10 times or less solution (10% by mass or more), and particularly preferably a 2 to 10 times solution (10 to 50% by mass). In the case of the cutting knife for potatoes, because the concentration of the adhered pathogens is extremely high (10⁷ to 10¹⁰ cfu/mL) and disinfection needs to be carried out in a short period of time at low temperatures, when the disinfectant is a 100 times or less solution, it is possible to sufficiently keep the sterilization capacity for the pathogens immediately after disinfection and completely sterilize the pathogens in a short period of time. In terms of completely inhibiting re-propagation of remaining pathogens, it is more preferable to use a 10 times or less solution. It should be noted that, because of the product design, it is preferable to be diluted to 2 times or more. When the disinfectant of the present invention is used, for example, the following method can be used. First, 7 L of tap water is placed in a bucket, and 1 kg of maleic acid is added thereto to make an 8 times solution. Maleic acid is completely dissolved, and subsequently, about 3 to 5 cutting knives (usually kitchen knives) are constantly immersed in the bucket. After one seed tuber is cut using one kitchen knife, another immersed kitchen knife is used sequentially (by using the cutting knife in this way, it is possible to prolong the time period for which the cutting knife is sterilized). When it takes one to two seconds to cut one seed tuber, 3 to 5 cutting knives are required for the sterilization. In this way, it becomes possible to obtain a minimum sterilization time period of 5 seconds or more per knife.

It should be noted that, after cutting the seed tuber by the above method, even when the pathogens adhere to the seed tuber in a stage before planting it in a farm field, the sterilization action continues on a cut surface of the seed tuber and the complete sterilization can be expected because the cutting knife has been treated with a maleic acid solution having a high concentration.

The disease control agent of the present invention can be widely applied to diseases. Examples of the disease on which the disease control agent particularly shows its effect include blackleg and soft rot of potato (scientific names of pathogens: Erwinia chrysanthemi, Erwinia cartovora var. cartovora, and the like), ring rot (scientific name of pathogen: Clavibacter michiganensis and the like), a bacterial damping-off of seeds of paddy rice and the like (scientific name of pathogen: Burkholderia plantarii and the like), a rice bacterial grain rot of seeds of paddy rice and the like (scientific name of pathogen: Burkholderia glumae and the like), a bacterial wilt of potato, tomato, and the like (scientific name of pathogen: Ralstonia solanacearum and the like), and diseases caused by filamentous fungi (scientific names of pathogens: Pythium ultimum, Fusarium oxysporum, Rhizoctonia solani, Pyricularia oryzae, Botrytis cinerea, and the like).

### EXAMPLES

The present invention will be described in more detail below with reference to Examples, but it goes without saying that the present invention is not limited to those examples only.

### Example 1

### Preparation of Disinfection Solution

### (1) Maleic Acid Solution

First, 1 kg of maleic acid was dissolved in 9 liters of tap water to make a 10% solution. This was defined as a maleic acid 10 times solution. In similar operations, maleic acid 2 times, 5 times, 25 times, 50 times, 100 times, 200 times, and 500 times solutions were made.

### (2) Maleic Anhydride Solution

First, 100 g of maleic anhydride was dissolved in 0.9 liter of acetone to make a 10% solution. This was defined as a maleic anhydride 10 times solution. In similar operations, maleic anhydride 2 times, 5 times, 25 times, 50 times, 100 times, 200 times, and 500 times solutions were made.

### Example 2

### Basic Test

### (Medium for Test)

Broth medium: 3 g of meat extract, 10 g of peptone, 5 g of NaCl, 1 L of water, pH 7.0 and 15 g of agar.

### (Preparation of Pathogen)

Erwinia carotovora var. cartovora was isolated from a potato (Mayqueen) diseased with blackleg and subjected to the test. Clavibacter michiganense subsp. Nebraskense (ATCC27822) which causes ring rot was purchased from American Type Culture Collection (ATCC) which is a biological resource center and used. The above pathogens were applied on the broth medium, cultured at 31° C. for two days, and the cultured pathogen were scraped away from the plate to prepare a microbial cell solution of 5×10⁹ cfu/mL. Pathogens were allowed to adhere to a cutting knife by immersing the cutting knife in this solution for 3 seconds.

### (Preparation of Test Solution)

As examples, used were the maleic acid 2 times, 5 times, 10 times, 25 times, 50 times, 100 times, 200 times, and 500 times solutions and the maleic anhydride 2 times, 5 times, 10 times, 25 times, 50 times, 100 times, 200 times, and 500 times solutions, which were made in Example 1 above. As a control, tap water was used. As Comparative Examples, Chemichlon G (granules of 70% calcium hypochlorite) 10 times solution, Vantocil (polyhexamethylene biguanide) 10 times solution, acetic acid 10 times and 50 times solutions, succinic acid 10 times and 50 times solutions, citric acid 10 times and 50 times solutions, and ascorbic acid 10 times and 50 times solutions were used.

### (Test Method)

A cutting knife to which the pathogens had been allowed to adhere was immersed in the above test solution for 5 seconds, and then washed thoroughly in 1 liter of sterilized water. The number of viable bacteria of the pathogens in this sterilized water was counted on the broth medium. The pathogens were cultured at 30° C. for 2 days.

### (Results)

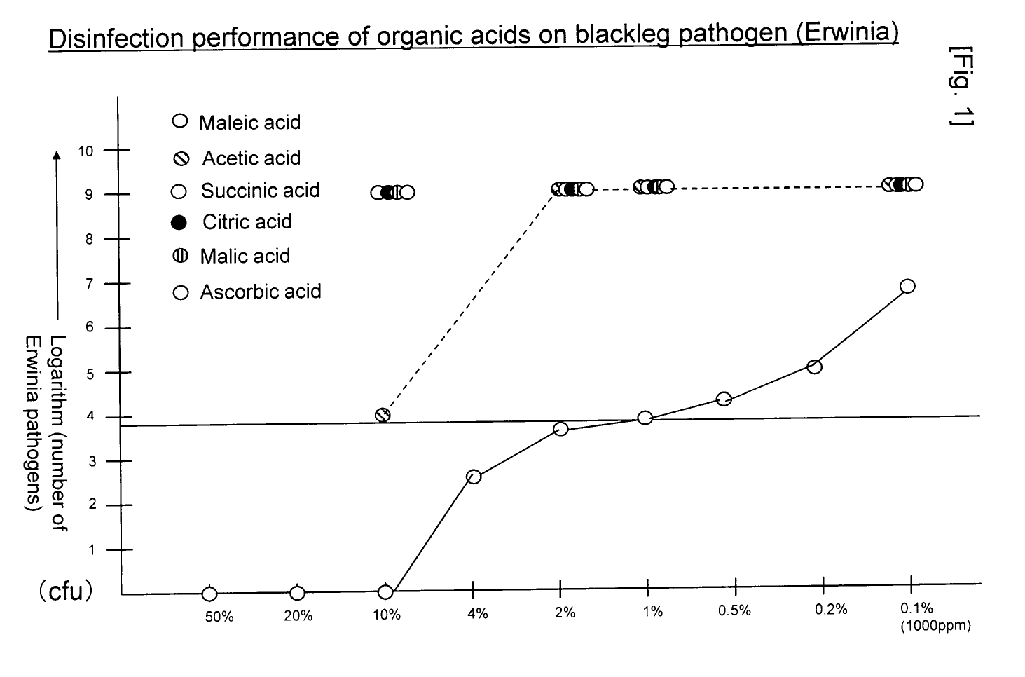

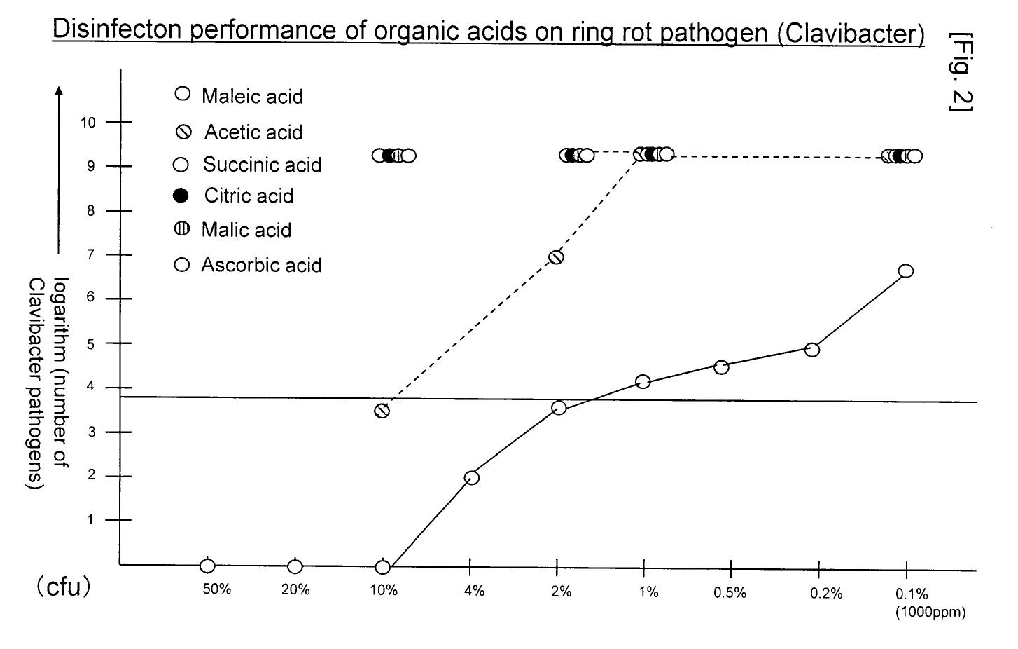

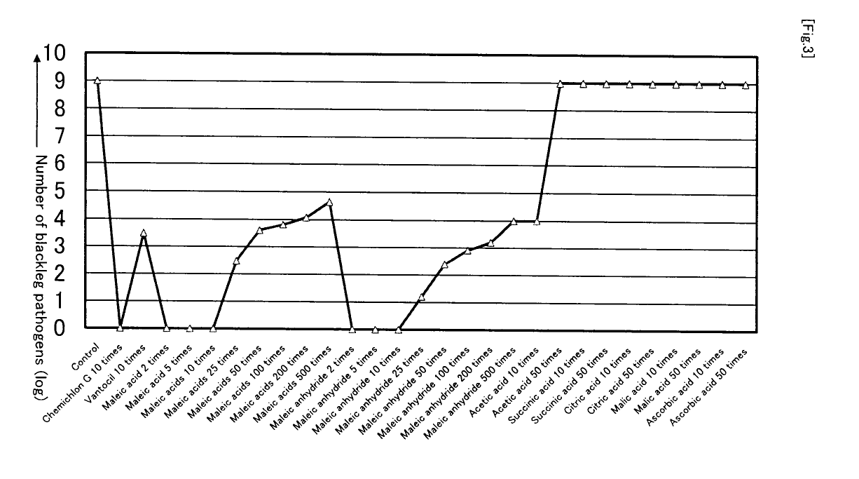

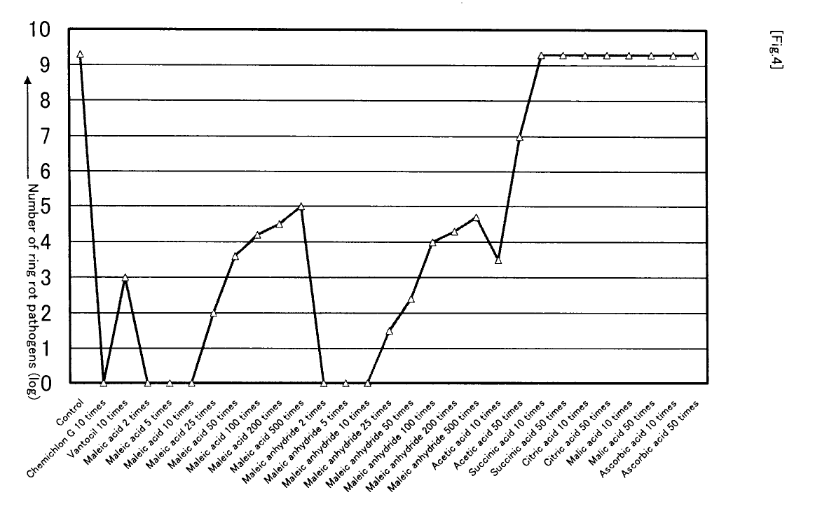

Results are shown in Table 1 (FIGS. 1 and 2), and FIGS. 3 and 4.

TABLE 1

| | | Logarithm (number | Logarithm (number |
|---|---|---|---|
| | | of blackleg | of ring rot |
| | Chemical | pathogens) (cfu) | pathogens) (cfu) |
| Control | | 9.0 | 9.3 |
| Comparative Example | Chemichlon G 10 times | 0.0 | 0.0 |
| Comparative Example | Vantocil 10 times | 3.5 | 3.0 |
| Example | Maleic acid 2 times | 0.0 | 0.0 |
| Example | Maleic acid 5 times | 0.0 | 0.0 |
| Example | Maleic acid 10 times | 0.0 | 0.0 |
| Example | Maleic acid 25 times | 2.5 | 2.0 |
| Example | Maleic acid 50 times | 3.6 | 3.6 |
| Example | Maleic acid 100 times | 3.8 | 4.2 |
| Example | Maleic acid 200 times | 4.1 | 4.5 |
| Example | Maleic acid 500 times | 4.7 | 5.0 |
| Example | Maleic anhydride 2 times | 0.0 | 0.0 |
| Example | Maleic anhydride 5 times | 0.0 | 0.0 |
| Example | Maleic anhydride 10 times | 0.0 | 0.0 |
| Example | Maleic anhydride 25 times | 1.2 | 1.5 |
| Example | Maleic anhydride 50 times | 2.4 | 2.4 |
| Example | Maleic anhydride 100 times | 2.9 | 4.0 |
| Example | Maleic anhydride 200 times | 3.2 | 4.3 |
| Example | Maleic anhydride 500 times | 4.0 | 4.7 |
| Comparative Example | Acetic acid 10 times | 4.0 | 3.5 |
| Comparative Example | Acetic acid 50 times | 9.0 | 7.0 |
| Comparative Example | Succinic acid 10 times | 9.0 | 9.3 |
| Comparative Example | Succinic acid 50 times | 9.0 | 9.3 |
| Comparative Example | Citric acid 10 times | 9.0 | 9.3 |
| Comparative Example | Citric acid 50 times | 9.0 | 9.3 |
| Comparative Example | Malic acid 10 times | 9.0 | 9.3 |
| Comparative Example | Malic acid 50 times | 9.0 | 9.3 |
| Comparative Example | Ascorbic acid 10 times | 9.0 | 9.3 |
| Comparative Example | Ascorbic acid 50 times | 9.0 | 9.3 |

As shown in Table 1 and FIGS. 1 and 2, it has been found that the complete sterilization is performed for blackleg and ring rot pathogens using the maleic acid and maleic anhydride 2 to 10 times solutions. It has been also shown that the maleic acid 25 to 50 times solutions and the maleic anhydride 25 to 50 times solutions also have a high sterilization effect.

### Example 3

### Test Using Potatoes

Erwinia carotovora var. cartovora cultured in Example 2 was inoculated to potatoes “Norin No. 1”, which was a breeder's stock of the potato, to make rotten potatoes. Under the condition of a room temperature of 5° C., the rotten potato was cut using a cutting knife. Subsequently, the cutting knife was immersed in the control (tap water), Comparative Examples (Chemichlon G 10 times solution or Vantocil 10 times solution) or Examples (maleic acid 5 times, 10 times, or 50 times solution, or maleic anhydride 5 times, 10 times, or 50 times solution) for 5 seconds. Then, five Norin No. 1 potatoes were cut using this cutting knife. The potatoes were kept humid for 2 days, and then planted in the farm field. A seeding was performed in early May, and fertilizer application and the management were performed according to a common practice. The effect was determined in early July, which was about two months after. Pathogenesis in stems above ground and a brown degree of ducts in the stem near a ground surface, which were characteristic for blackleg, were evaluated as a pathogenesis degree (ratio of an area where the pathogenesis was observed to the total area of the stem) in average of 5 potatoes. The results are shown in Table 2.

TABLE 2

| | | Degree | Chemical |
|---|---|---|---|
| | | of pathogenesis | injury |
| Control | Tap water | 5 | — |
| Comparative | Chemichlon G 10 times | 0 | — |
| Example | | | |
| Comparative | Vantocil 10 times | 3 | — |
| Example | | | |
| Example | Maleic acid 5 times | 0 | — |
| Example | Maleic acid 10 times | 0 | — |
| Example | Maleic acid 50 times | 3 | — |
| Example | Maleic anhydride 5 times | 0 | — |
| Example | Maleic anhydride 10 times | 0 | — |
| Example | Maleic anhydride 50 times | 2 | — |

*Degree of pathogenesis
0: No pathogenesis
1: 5% or less
2: 5 to less than 20%
3: 20 to less than 40%
4: 40 to less than 70%
5: 70% or more

It has been found that the complete sterilization is performed using maleic acid 5 to 10 times solution and maleic anhydride 5 to 10 times solution. It has also been shown that maleic acid 50 times and maleic anhydride 50 times solutions have a high sterilization effect.

### Example 4

### Material Disinfection Test

As pathogens for bacterial diseases, a bacteria of potato blight (Erwinia chrysanthemi), a bacteria causing damping-off of seeds of paddy rice (Burkholderia plantarii), a bacteria causing grain rot of seeds of paddy rice (Burkholderia glumae), and a bacterial pathogen causing wilt of tomato (Ralstonia solanacearum) were used.

The above pathogens were applied on the broth medium, cultured at 31° C. for two days, and the cultured pathogens were scraped away from the plate to prepare 500 cc of solutions each of 1×10⁹ cfu/mL.

Pythium ultimum, Fusarium oxysporum, Rhizoctonia solani, Pyricularia oryzae, and Botrytis cinerea as filamentous fungi pathogens were cultured on a potato dextrose medium in the plate at 25° C. to induce sporulation. Spores were scraped away from the plate to prepare 500 cc of solutions each of 1×10⁶ cfu/mL.

As the material to be disinfected, a raising seedling box (wooden box or plastic box), a raising seedling pot, an orange storage box, a plow, a sickle, a harrow, a saw, a chopper, a kitchen knife, a knife, a sword, a rake, or a shovel was used, thoroughly washed with water to make a stain go away and thoroughly dried in the shade for 3 hours before spraying the pathogenic solution.

Solutions of the aforementioned pathogens for bacterial diseases and filamentous fungi disease were evenly sprayed on the thoroughly dried material using a spray atomizer, and then the material was left to stand for 15 minutes.

As a disinfectant for comparative examples, 500 cc of each of a 1,000 times solution of Chemichlon G, a 500 times solution of Ichiban, a 10 times and a 50 times solution of acetic acid, a 10 times and a 50 times solution of succinic acid, a 10 times and a 50 times solution of citric acid, and a 10 times and a 50 times solution of ascorbic acid were produced. As disinfectants for examples, 500 cc of each of a 2 times, 5 times, 25 times, 50 times, 100 times, 200 times, and 500 times solutions of maleic acid were produced according to the method of Example 1. In addition, 500 cc of each of 2 times, 5 times, 25 times, 50 times, 100 times, 200 times, and 500 times solutions of maleic anhydride were produced according to Example 1.

The above disinfectant was evenly sprayed on the above material 15 minutes after spray of the pathogens. 5 minutes after spray of the disinfectant to each material, the surface of each material was wiped with a cotton swab wetted with sterilized water in five random points on the material. The wiped cotton swab was thoroughly washed in 10 mL of sterilized water. This solution was serially diluted by 10 times to 10⁸ times. Subsequently, 0.1 mL of each of the diluted solutions of 10 to 10⁸ times of the pathogens for bacterial diseases and the filamentous fungi disease was seeded on the medium, cultured at 25° C. for 3 days for the filamentous fungi and 30° C. for 2 days for the bacteria. Then the number of viable pathogens was counted. The bacteria were counted on the broth medium and the filamentous fungi were counted on the potato dextrose medium. An average value of the viable pathogens in the five points on each material was calculated, and from the average value, an average value of the viable pathogens in all materials examined was calculated. The effect of each disinfectant is shown by this value. This value was obtained for each pathogen. The results are shown in Table 3.

TABLE 3
Disinfection effect on bacteria pathogens (Average value of
viable bacteria counts on all materials examined: cfu/ml)

| | Erwinia | Burkholderia | Burkholderia | Ralstonia |
|---|---|---|---|---|
| | chrysanthemi | plantarii | glumae | solanacearum |
| Tap water | 1.5 × 10⁸ | 6.5 × 10⁷ | 2.7 × 10⁸ | 1.0 × 10⁹ |
| Chemichlon G 1,000 times | 0 | 0 | 0 | 0 |
| Ichiban 500 times | 0 | 0 | 0 | 0 |
| Maleic acid 2 times | 0 | 0 | 0 | 0 |
| Maleic acid 5 times | 0 | 0 | 0 | 0 |
| Maleic acid 25 times | 0 | 0 | 0 | 0 |
| Maleic acid 50 times | 0 | 0 | 0 | 0 |
| Maleic acid 100 times | 0 | 0 | 0 | 0 |
| Maleic acid 200 times | 53 | 65 | 93 | 18 |
| Maleic acid 500 times | 3.5 × 10³ | 1.5 × 10² | 5.0 × 10³ | 1.0 × 10² |
| Maleic anhydride 2 times | 0 | 0 | 0 | 0 |
| Maleic anhydride 5 times | 0 | 0 | 0 | 0 |
| Maleic anhydride 25 times | 0 | 0 | 0 | 0 |
| Maleic anhydride 50 times | 0 | 0 | 0 | 0 |
| Maleic anhydride 100 times | 0 | 0 | 0 | 0 |
| Maleic anhydride 200 times | 11 | 7 | 69 | 4 |
| Maleic anhydride 500 times | 1.3 × 10² | 1.0 × 10² | 2.0 × 10² | 58 |
| Acetic acid 10 times | 3.2 × 10³ | 1.2 × 10³ | 5.0 × 10² | 2.8 × 10² |
| Acetic acid 50 times | 1.1 × 10⁷ | 8.3 × 10⁶ | 3.3 × 10⁷ | 6.0 × 10⁸ |
| Succinic acid 10 times | 1.0 × 10⁸ | 2.5 × 10⁷ | 1.0 × 10⁸ | 1.1 × 10⁹ |
| Succinic acid 50 times | 1.0 × 10⁸ | 2.6 × 10⁷ | 1.5 × 10⁸ | 8.5 × 10⁸ |
| Citric acid 10 times | 1.7 × 10⁸ | 3.8 × 10⁷ | 2.6 × 10⁸ | 1.1 × 10⁹ |
| Citric acid 50 times | 1.9 × 10⁸ | 1.0 × 10⁷ | 1.7 × 10⁸ | 1.5 × 10⁹ |
| Malic acid 10 times | 1.4 × 10⁸ | 1.0 × 10⁷ | 3.6 × 10⁸ | 9.5 × 10⁸ |
| Malic acid 50 times | 1.2 × 10⁸ | 3.2 × 10⁷ | 5.2 × 10⁸ | 7.8 × 10⁸ |
| Ascorbic acid 10 times | 1.0 × 10⁸ | 1.6 × 10⁷ | 1.1 × 10⁸ | 8.5 × 10⁸ |
| Ascorbic acid 50 times | 1.0 × 10⁸ | 1.8 × 10⁷ | 3.2 × 10⁸ | 1.0 × 10⁹ |

Disinfection effect on filamentous fungi pathogens (average
value of viable filamentous fungi counts on all materials
examined: cfu/ml)

| | Pythium | Fusarium | Rhizoctonia | Pyricularia | Botrytis |
|---|---|---|---|---|---|
| | ultimum | oxysporum | solani | oryzae | cinerea |
| Tap water | 4 × 10⁴ | 5.74 × 10⁵ | 2.5 × 10⁵ | 7.5 × 10⁴ | 5.5 × 10⁵ |
| chemichlon G 1,000 times | 0 | 0 | 0 | 0 | 0 |
| Ichiban 500 times | 0 | 0 | 0 | 0 | 0 |
| Maleic acid 2 times | 0 | 0 | 0 | 0 | 0 |
| Maleic acid 5 times | 0 | 0 | 0 | 0 | 0 |
| Maleic acid 25 times | 0 | 0 | 0 | 0 | 0 |
| Maleic acid 50 times | 0 | 0 | 0 | 0 | 0 |
| Maleic acid 100 times | 0 | 0 | 0 | 0 | 0 |
| Maleic acid 200 times | 73 | 1.1 × 10² | 2.5 × 10² | 1.5 × 10² | 1.7 × 10² |
| Maleic acid 500 times | 3.1 × 10³ | 7.8 × 10² | 3.1 × 10² | 2.7 × 10² | 1.0 × 10² |
| Maleic anhydride 2 times | 0 | 0 | 0 | 0 | 0 |
| Maleic anhydride 5 times | 0 | 0 | 0 | 0 | 0 |
| Maleic anhydride 25 times | 0 | 0 | 0 | 0 | 0 |
| Maleic anhydride 50 times | 0 | 0 | 0 | 0 | 0 |
| Maleic anhydride 100 times | 0 | 0 | 0 | 0 | 0 |
| Maleic anhydride 200 times | 15 | 56 | 98 | 32 | 65 |
| Maleic anhydride 500 times | 1.1 × 10³ | 2.0 × 10² | 1.1 × 10² | 1.5 × 10² | 1.1 × 10² |
| Acetic acid 10 times | 58 | 76 | 33 | 98 | 65 |
| Acetic acid 50 times | 6.8 × 10³ | 4.2 × 10² | 3.1 × 10² | 1.5 × 10² | 2.6 × 10² |
| Succinic acid 10 times | 7.8 × 10³ | 2.3 × 10⁵ | 1.5 × 10⁵ | 3.2 × 10⁴ | 3.5 × 10⁵ |
| Succinic acid 50 times | 3.0 × 10⁴ | 4.5 × 10⁵ | 2.1 × 10⁵ | 3.1 × 10⁴ | 4.3 × 10⁵ |
| Citric acid 10 times | 2.8 × 10⁴ | 3.7 × 10⁵ | 2.0 × 10⁵ | 2.8 × 10⁴ | 2.6 × 10⁵ |
| Citric acid 50 times | 1.7 × 10⁴ | 3.4 × 10⁵ | 1.7 × 10⁵ | 3.2 × 10⁴ | 4.6 × 10⁵ |
| Malic acid 10 times | 2.5 × 10⁴ | 1.8 × 10⁵ | 2.1 × 10⁵ | 4.6 × 10⁴ | 3.7 × 10⁵ |
| Malic acid 50 times | 4.3 × 10⁴ | 4.5 × 10⁵ | 1.8 × 10⁵ | 5.0 × 10⁴ | 2.9 × 10⁵ |
| Ascorbic acid 10 times | 2.3 × 10⁴ | 3.2 × 10⁵ | 1.3 × 10⁵ | 4.2 × 10⁴ | 4.5 × 10⁵ |
| Ascorbic acid 50 times | 1.5 × 10⁴ | 4.7 × 10⁵ | 2.0 × 10⁵ | 6.2 × 10⁴ | 5.0 × 10⁵ |

As is evident from the results in Table 3, it is obvious that the complete sterilization is possible by disinfecting with maleic acid and maleic anhydride at a dilution ratio of 2 to 100 times for 5 minutes.

### INDUSTRIAL APPLICABILITY

The disinfectant of the present invention is useful as a microbicide for disinfecting the materials such as raising seedling boxes (wooden boxes, plastic boxes), raising seedling pots, pot braces, fruit storage boxes, and farm tools in the agricultural field. In particular, the disinfectant of the present invention is useful for disinfecting the farm tools. The disinfectant of the present invention is also useful for preventing blackleg and soft rot (caused by the pathogens belonging to the genus Erwinia) and ring rot, which transmit through a cutting knife upon cutting a seed tuber of a potato, by disinfecting the cutting knife.

For the disinfection of the ordinary agricultural materials, e.g., the raising seedling boxes (wooden boxes, plastic boxes), the raising seedling pots, the pot braces, the fruit storage boxes, the farm tools, and the like, a disinfection solution prepared by diluting maleic acid alone or together with a surfactant with tap water to a 50 to 500 times solution can be used for sufficient sterilization. Neither protection glasses nor face guard is necessary upon use of the disinfectant, and the material can be used immediately after disinfection, and the remaining disinfection solution can be discarded into the environment without any special spherical treatment because the solution rapidly degrades in the environment.

When it is necessary to disinfect the material to which the pathogens have adhered at an extremely high concentration (e.g., 10⁹ cfu/mL or more), a disinfection solution prepared by diluting maleic acid or maleic anhydride alone or together with a surfactant, with tap water or an organic solvent such as acetone or DMSO to a 2 to 10 times solution is typically used. For example, when the above disinfectant is used, Erwinia bacteria of 10⁹ cfu/mL or more adhering to the cutting knife for potatoes can be completely sterilized at a temperature of as low as 2 to 8° C. in an extremely short period of time of 5 seconds or less. Maleic acid is easily soluble in water, and can be used for cutting of potatoes by the manual labor or by incorporating into a seed tuber planting apparatus. Maleic anhydride is poorly soluble in water, but if it is dissolved in acetone, DMSO, or the like, it can be used as the disinfectant in the same way as maleic acid.

1. A disinfectant comprising as an active ingredient maleic acid and/or maleic anhydride, wherein a content of the maleic acid and/or the maleic anhydride is 1% by mass or more with respect to an entire disinfectant.

2. The disinfectant according to claim 1, which is an agent for controlling an agricultural disease.

3. The disinfectant according to claim 1, which is an agent for disinfecting a cutting knife.

4. The disinfectant according to any of claims 1 to 3, wherein the disease is caused by any one of bacteria, filamentous fungi, actinomycetes, and viruses.

5. A method of controlling a disease, comprising treating a cutting knife with the disinfectant according to claim 3.

6. The method of controlling a disease according to claim 5, wherein the disease is blackleg, soft rot, or ring rot of potatoes.

7. The method of controlling a disease according to claim 6, wherein the blackleg or soft rot is caused by any one of Erwinia chrysanthemi and Erwinia carotovora var. carotovora, and the ring rot is caused by Clavibacter michiganensis.

8. A method of controlling a disease, comprising disinfecting an agricultural material with maleic acid and/or maleic anhydride.

9. Use of maleic acid and/or maleic anhydride for disinfection.
